(19) United States
(12) Patent Application Publication
(10) Pub. No.: US 2008/0207998 A1
(43) Pub. Date: Aug. 28, 2008

(54) ENDOSCOPE APPARATUS

(75) Inventor: Koji MARUYAMA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/037,512

(22) Filed: Feb. 26, 2008

(30) Foreign Application Priority Data
Feb. 28, 2007 (JP) 2007-050631

(51) Int. Cl. A61B 1/01

(52) U.S. Cl. 600/114; 600/146

(57) ABSTRACT

An endoscope apparatus includes an operation section main body configured to include a frame body and bumper members provided to cover plural surfaces of the frame body. An operation section is configured to include the operation section main body and a grasping section. An endoscope is configured to include an insertion section extended out from one surface side of the operation section main body and including a bending section in a position more proximal than a distal end and a universal cable extended out from the other surface side of the operation section main body opposed to the one surface. On the other hand, the universal cable is connected to an apparatus main body.

This application claims benefit of Japanese Application No. 2007-050631 filed in Japan on Feb. 28, 2007, the contents of which are incorporated by this reference.

### BACKGROUND OF THE INVENTION

1. Field of the Invention

The present invention relates to an endoscope apparatus including an endoscope in which an insertion section is extended out from an operation section and an apparatus main body to which the endoscope is detachably attachable.

2. Description of the Related Art

Endoscopes are widely used in the medical field and the industrial field. With the endoscope used in the medical field, it is possible to observe organs in a body cavity by inserting a thin and long insertion section into the body cavity and perform various treatments and tests by inserting a treatment instrument into the body cavity through an insertion channel as required.

On the other hand, with the endoscope used in the industrial field, it is possible to perform observation of presence or absence of flaws in an inspection area, or presence or absence of corrosion and the like, various kinds of repair, or the like by inserting a thin and long insertion section into a jet engine, pipes of a factory, and the like.

Some industrial endoscope apparatus is configured by an endoscope having a thin and long insertion section, for example, at a distal end of which an image pickup unit having an image pickup lens, an image pickup device such as a CCD, and a light-emitting element such as an LED is disposed, and an apparatus main body to which the endoscope is connected.

For example, Japanese Patent Laid-Open No. 2003-325437 discloses a drawing member operating apparatus with which it is possible to operate to tilt an operation indication lever with a slight amount of control force and move a desired drawing member by a desired amount to perform bending operation for a bending section. In the drawing member operating apparatus, since an operator grasps a grasping section to handle an endoscope, an arrangement example or the like of the grasping section is shown taking into account graspability. However, installability of the endoscope is not taken into account.

Japanese Patent Laid-Open No. 2004-81797 discloses an endoscope apparatus excellent in graspability and operability with which a burden on an arm of an operator who grasps an endoscope is reduced and the operator can freely perform twisting operation for an insertion section and bending operation for a bending section.

In the endoscope apparatus, the operator can handle the endoscope in a state in which the operator wears an apparatus main body as shown in FIG. 19 of Japanese Patent Laid-Open No. 2004-81797 or handle the endoscope in a state in which the apparatus main body is placed on a surface of a work desk or the like as shown in FIG. 20. In the endoscope apparatus, plural legs are provided on a bottom surface of an operation section main body and a bottom surface of a grasping section taking into account grippability and stable installability as shown in FIG. 16A and FIG. 16B of Japanese Patent Laid-Open No. 2004-81797. The legs are made of rubber and formed in a predetermined shape.

### SUMMARY OF THE INVENTION

An endoscope apparatus includes an operation section main body configured to include a frame body and bumper members provided to cover plural surfaces of the frame body, an operation section configured to include the operation section main body and a grasping section, an endoscope including an insertion section extended out from one surface side of the operation section main body and including a bending section in a position more proximal than a distal end and a universal cable extended out from the other surface side of the operation section main body opposed to the one surface, and an apparatus main body to which the universal cable is connected.

The above and other objects, features and advantages of the invention will become more clearly understood from the following description referring to the accompanying drawings.

### BRIEF DESCRIPTION OF THE DRAWINGS

FIG. 1 is a diagram for explaining an endoscope apparatus configured by an endoscope and an apparatus main body including a monitor;

FIG. 2 is a diagram for explaining a configuration of the endoscope including a perspective view of an operation section;

FIG. 3 is a diagram for explaining operation section housing sides and bumper members configuring side configuring sections of the operation section;

FIG. 4 is a front view of the operation section;

FIG. 5 is a perspective view for explaining an internal configuration of the operation section;

FIG. 6 is a diagram for explaining a bending mechanism provided in the inside of the operation section;

FIG. 7 is a side view of the operation section vertically placed on a surface; and

FIG. 8 is a perspective view showing the vertically-placed operation section and the apparatus main body.

### DETAILED DESCRIPTION OF THE PREFERRED EMBODIMENTS

An embodiment of the present invention will be hereinafter explained with reference to the drawings.

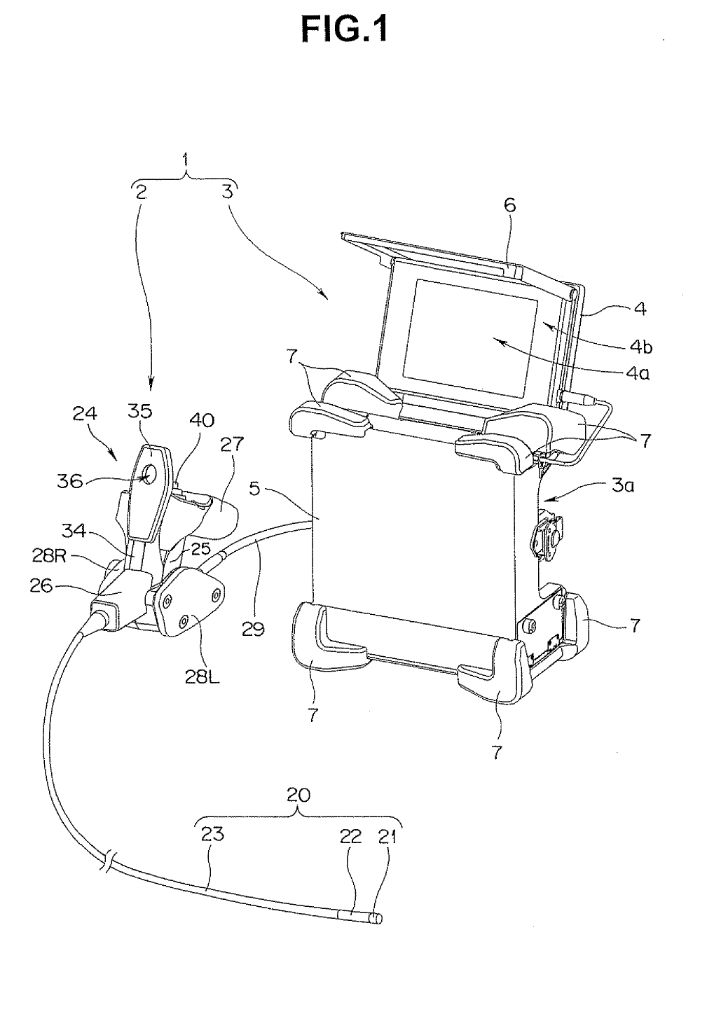

As shown in FIG. 1, an endoscope apparatus 1 is configured to include an endoscope 2 and an apparatus main body 3 to which the endoscope 2 is electrically connected.

The endoscope 2 is, for example, an industrial endoscope of a battery driving type incorporating an optical lens, an image pickup device (not shown), a light-emitting element, and the like at a distal end portion 21 of an insertion section 20. The apparatus main body 3 is configured to include an image processing section, a monitor 4 as a display device, and a battery (not shown) which supplies electric power. The image processing section performs processing such as driving of the image pickup device and generation of a video signal from an image signal outputted from the imaging element. The monitor 4 receives the video signal outputted from the image processing section and displays an endoscopic image on an image display surface 4a. The battery supplies electric power to the light-emitting element, the monitor 4, and the like.

As the image pickup device incorporated in the distal end portion 21, for example, a C-MOS (complementary metal oxide semiconductor) or the like is used As the light-emitting element, for example, an LED (light-emitting diode) or the like is used. A bending section 22 is configured by coupling not-shown joint rings and configured to be capable of bending in a predetermined direction. The bending section 22 according to the present embodiment is configured to be capable of bending (in four directions) up and down and to the left and right.

The apparatus main body 3 is formed in, for example, a box shape. The monitor 4 is fixed to an armor housing 5 configuring the apparatus main body 3 via hinges (not shown) or the like. In the present embodiment, the monitor 4 is openable and closable and is configured such that, in a closed state, a rear side of a monitor surface 4a having the image display surface 4b is arranged on a back side in FIG. 1 of the armor housing 5.

Reference numeral 6 denotes a cover, which covers and protects the image display surface 4a when the endoscope apparatus 1 is not used. Reference numeral 7 denotes legs, which are formed by an elastic member such as NBR (nitrile butadiene rubber) and attached to all corners of the armor housing 5. The legs 7 are shock absorbing members which configure protectors for absorbing a shock given to the armor housing 5 and protecting the armor housing 5 and absorb and relax the shock to the armor housing 5. The apparatus main body 3 can be set in vertical placement in which the legs 7 are placed on the ground, a floor surface, or the like and the image display surface 4a of the monitor 4 in, for example, the closed state are arranged substantially vertically to the ground or in horizontal placement in which the image display surface 4a of the monitor 4 in the closed state is arranged substantially parallel to the ground.

The endoscope 2 is configured to include the insertion section 20, an operation section 24, and a universal cable 29 having flexibility. The universal cable 29 extends out from the operation section 24. The insertion section 20 is thin and long and has flexibility, and is configured by coupling, in order from a distal end side, the distal end portion 21, the bending section 22 bendable in the up and down directions and the left and right directions, and a flexible tube section 23 having flexibility. The operation section 24 is coupled to a proximal end portion of the insertion section 20. In the universal cable 29, an electric cable (not shown) extended out from the apparatus main body 3 to the light-emitting element of the distal end portion 21, a signal cable (not shown) through which a driving control signal of the image pickup device or an image signal photoelectrically converted by the image pickup device is transmitted, and the like are inserted. A connector box 29a detachably attachable to a connector box housing chamber 3a provided in the apparatus main body 3 is provided at a proximal end portion of the universal cable 29.

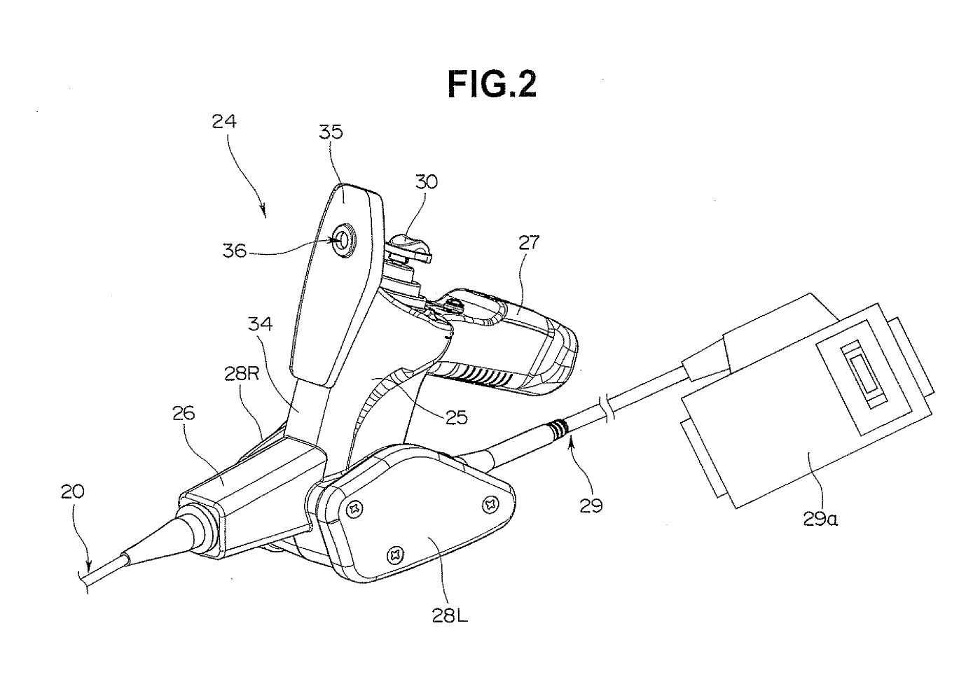

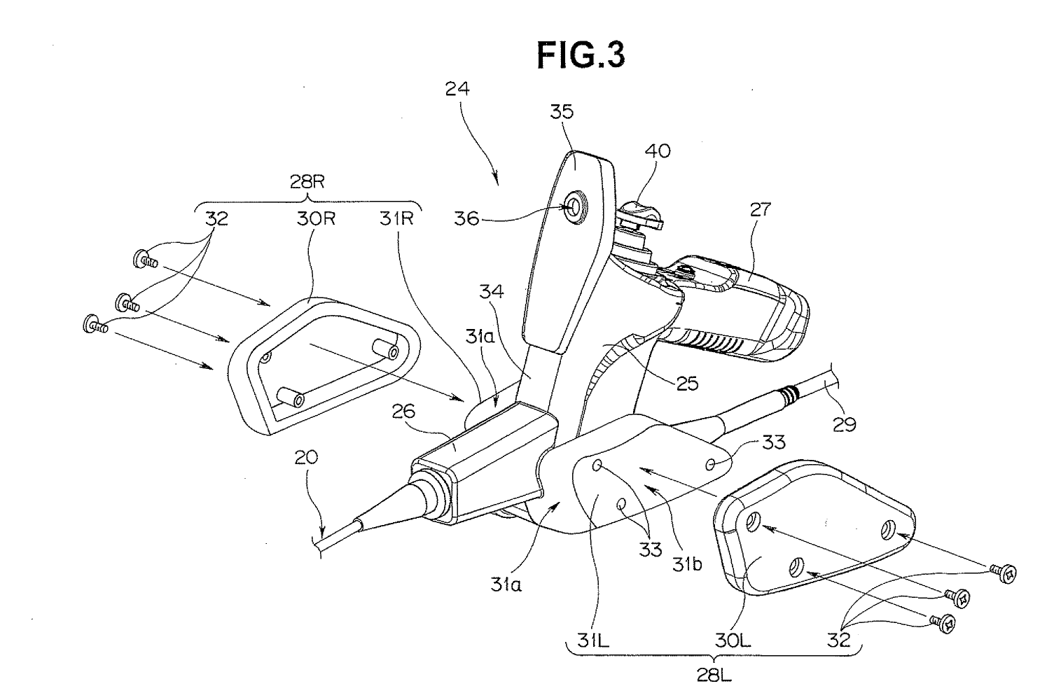

As shown in FIGS. 1 and 2, the operation section 24 is configured to include an operation section main body 25, an operation wire disposing section 26, a grasping section 27, and side configuring sections 28L and 28R. As shown in FIG. 3, the operation section main body 25 is configured to include a frame body portion and bumper members. The side configuring sections 28L and 28R are configured by fixing, with screws 32, bumper members 30L and 30R formed in symmetrical shapes to sides 31L and 31R of an operation section housing as a frame body (hereinafter referred to as operation section housing sides 31L and 31R). In other words, outer peripheral portions 31a and side portions 31b of the operation section housing sides 31L and 31R are covered with the bumper members 30. Reference numeral 33 denotes female screw portions in which the screws 32 are screwed.

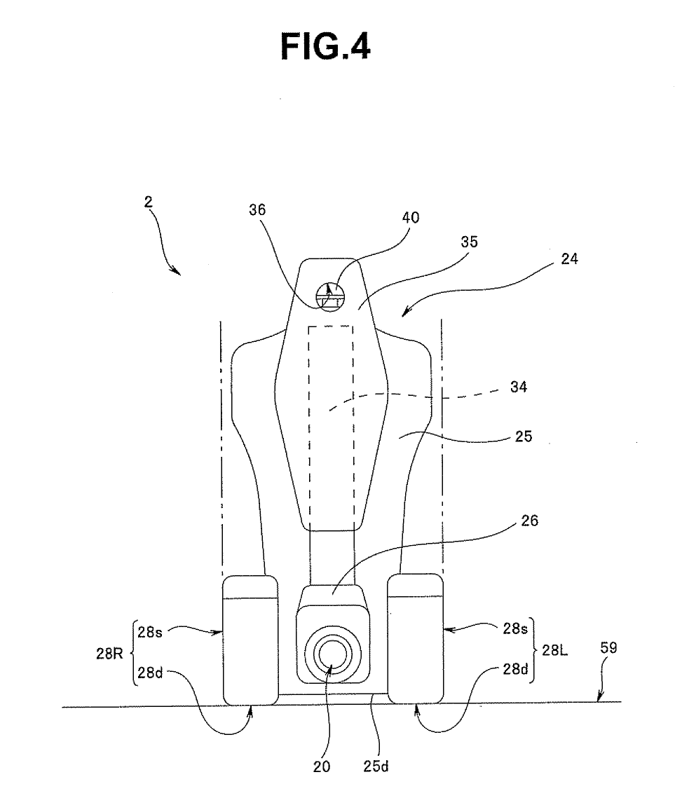

The bumper members 30L and 30R are shock absorbing members which absorb a shock and relax a load applied to the operation section 24. When the operation section 24 is placed or when a careless drop or the like occurs, the bumper members 30L and 30R absorb a shock load. Specifically, the bumper members 30L and 30R are formed in a block shape by, for example, rubber, alpha gel, resin, or a foaming member containing an elastic member such as NBR (nitrile butadiene rubber). The bumper members 30L and 30R are provided to project further than outer surfaces of the outer peripheral portions 31a and outer surfaces of the side portions 31b of the operation section housing sides 31L and 31R. Therefore, as shown in FIG. 4, setting surfaces 28d, which are lower surfaces of the side configuring sections 28L and 28R, protrude further than a lower surface 25d of the operation section main body 25.

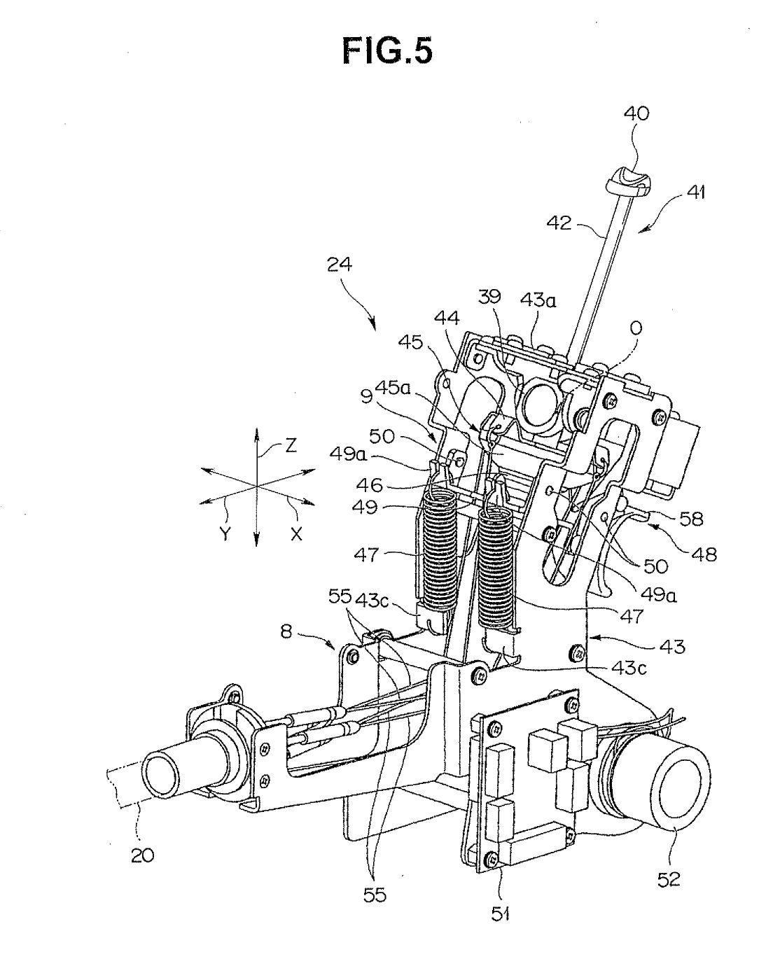

A bending lever 40 configuring a drawing operation member 41 (see FIG. 5) described later is protrudingly provided on a top surface (or an upper surface) of the operation section main body 25. The drawing operation member 41 is an operation indicating mechanism for causing the bending section 22 to perform a bending operation.

Sides of the bumper members 30L and 30R project further than sides of the operation section main body 25 as indicated by alternate long and two shot dashes lines.

An internal configuration of the operation section 24 is explained.

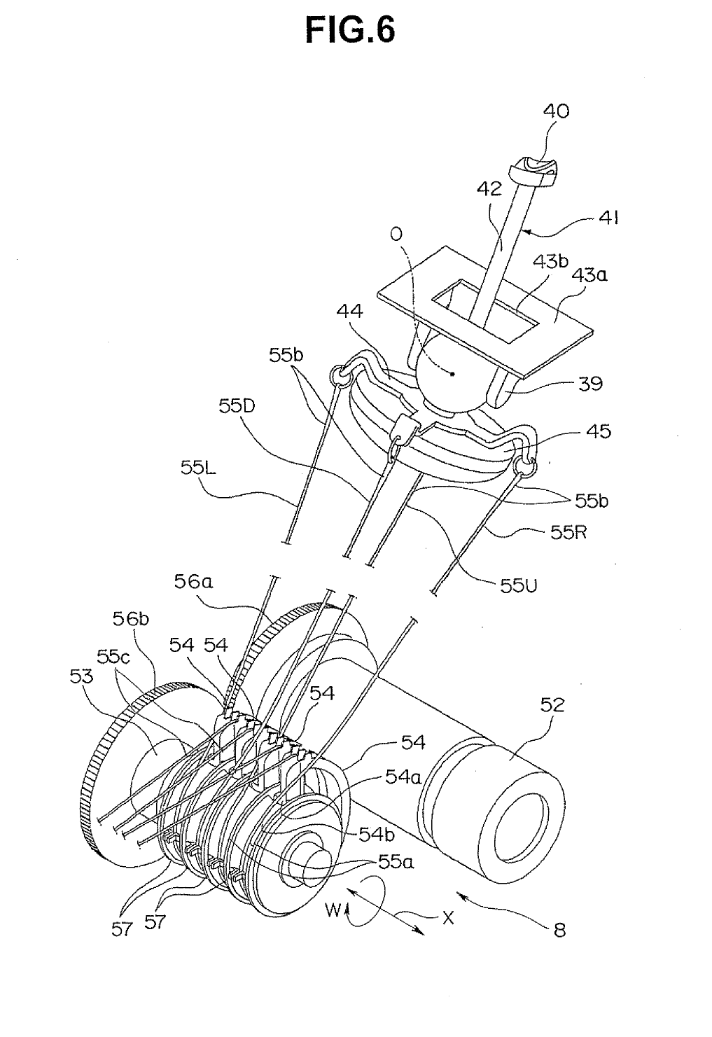

The internal configuration of the operation section 24 is explained with reference to FIGS. 5 and 6. In the present embodiment, an X direction shown in FIG. 5 is set as a left to right direction, a Y direction shown in FIG. 5 is set as a front to rear direction, and a Z direction shown in FIG. 5 is set as an up to down direction. In other words, the insertion section 20 is provided to extend out from the front of the operation section 24, the bending lever 40 is provided above the operation section 24, and a substrate 51 described later is provided in a left direction of the operation section 24.

In the operation section 24 shown in FIG. 5, a bending mechanism 8 for bending the bending section 22 shown in FIG. 1 and a lock mechanism 9 for fixing the movement of the bending mechanism 8 are provided.

As shown in FIG. 6, the bending mechanism 8 is configured by a driving motor 52, a pulley 53, plural annular members (hereinafter described as C rings) 54, plural operation wires 55 as drawing members, and the drawing operation member 41. The driving motor 52 is a driving device which drives to rotate a not-shown driving shaft. The pulley 53 is configured to be rotatable by a rotation driving force of the driving motor 52. The C rings 54 are externally attached to the pulley 53. In the present embodiment, there are four C rings 54. The operation wires 55 are wound around the respective C rings 54. In the present embodiment, the drawing operation member 41 operates to draw the four operation wires 55.

The driving motor 52 is one of heavy objects. As shown in FIGS. 5 and 6, the driving motor 52 is disposed in a lower part and a side of the operation section 24. A first gear 56a is provided in the driving shaft of the driving motor 52. The driving motor 52 is provided through a frame 43, which is formed by two plate members arranged to be opposed to each other, and fixed to the frame 43.

As shown in FIG. 6, the pulley 53, which is one of heavy objects, is disposed in a lower part of the operation section 24 and on a front side of the driving motor 52. At one end of the pulley 53, a second gear 56b which meshes with the first gear 56a is provided. A rotation driving force of the driving motor 52 is transferred to the pulley 53 via the first gear 56a and the second gear 56b. The pulley 53 axially rotates as indicated by an arrow W. The pulley 53 is disposed in the frame 43 shown in FIG. 5 and attached to the frame 43.

As shown in FIG. 6, the C rings 54 are elastically deformable members formed in a substantially C shape. The respective C rings 54 are externally attached to the pulley 53 rotatably. The C rings 54 and the operation wires 55 form pairs and are provided side by side in an X direction, which is an axial direction of the pulley 53. Substantially annular spacers 57 are interposed among the C rings 54 and the C rings 54 adjacent to one another, respectively. The spacers 57 are externally attached to the pulley 53.

As shown in FIG. 6, middle portions 55a of the respective operation wires 55 are wound around outer peripheral surfaces 54b of the respective C rings 54 which form pairs with the operation wires 55. Proximal end portions 55b of the respective operation wires 55 are connected to the drawing operation member 41. On the other hand, not-shown distal end portions of the respective operation wires 55 are connected to respective bending dies, which configure the bending section 22, in the inside of the insertion section 20 shown in FIG. 1, respectively.

The respective operation wires 55 are wound around to turn the middle portions 55a around the C rings 54 such that the proximal end portions 55b connected to the drawing operation member 41 and the distal end portion connected to the bending section 22 cross-viewed from a side. Consequently, by drawing the proximal end portions 55b, it is possible to tighten the C rings 54 against the pulley 53.

The four operation wires 55 described above are, specifically, an upward operation wire 55U, a downward operation wire 55D, a leftward operation wire 55L, and a rightward operation wire 55R shown in FIG. 6. The proximal end portions 55b of the operation wires 55 are fixed to an end of a supporting plate 44 provided in the drawing operation member 41, respectively. Specifically, the upward operation wire 55U and the downward operation wire 55D are fixed to the supporting plate 44 in the positions opposed to each other. The leftward operation wire 55L and the rightward operation wire 55R are fixed to be opposed to each other in a direction substantially orthogonal to a direction in which the upward operation wire 55U and the downward operation wire 55D are fixed.

As shown in FIGS. 5 and 6, the drawing operation member 41 is configured by a bar-like operation lever 42 as an indication member, a universal joint 39, and the supporting plate 44. The universal joint 39 is provided in the middle of the operation lever 42. The supporting plate 44 is provided at a lower end of the operation lever 42. The operation lever 42 is inserted through a rectangular opening 43b of a frame member 43a provided at an upper end of the frame 43 of the operation section 24. An upper part of the operation lever 42 is projected from the opening 43b described above to the outside of the frame 43. The operation lever 42 is attached to the frame 43 via the universal joint 39. The drawing operation member 41 is supported rotatably to the front, the rear, the left, and the right around a rotation center point O of the universal joint 39. The supporting plate 44 is a plate material of a substantially cross shape and vertically attached to the lower end of the operation lever 42. The proximal end portions 55b of the respective operation wires 55 are attached to respective ends in the four direction of the supporting plate 44, respectively.

As shown in FIGS. 5 and 6, the lock mechanism 9 is a mechanism for closely attaching a stopper 46 to a spherical surface 45a of a fixed section 45 provided on the drawing operation member 41 side and regulating the movement of the rotatable drawing operation member 41. The lock mechanism 9 is configured to roughly include the fixed section 45 provided on the drawing operation member 41 side, the stopper 46 closely attached to the spherical surface 45a of the fixed section 45, for example, a pair of urging members 47 which urge the stopper 46, and a moving mechanism 48 which moves the stopper 46.

The stopper 46 is fixed in a state in which the stopper 46 is placed on an upper surface of a tabular stopper supporting plate 49. The stopper supporting plate 49 is attached to the frame 43 rotatably in the vertical direction. Specifically, reference numerals 50 and 50 denote rotating shafts. The rotating shafts 50 are fixed to the frame 43 vertically rotatably. The stopper 46 is disposed in a substantially center position of the stopper supporting plate 49. The rotating shafts 50 and 50 of the stopper supporting plate 49 are provided in positions closer to the front of the stopper supporting plate 49 in the present embodiment.

As shown in FIG. 5, the pair of urging members 47 are members for pressing a contact surface of the stopper 46 against the spherical surface 45a of the fixed section 45. The urging members 47 according to the present embodiment are helical springs extending in the up to down direction. Lower ends of the urging members 47 and 47 are locked by locked sections 43c and 43c formed in the frame 43, respectively. Upper ends of the urging members 47 are locked by locked sections 49a and 49a formed in the stopper supporting plate 49, respectively. The urging members 47 urge the locked sections 49a and 49a of the stopper supporting plate 49 in a direction for pulling down the locked sections 49a and 49a. The stopper supporting plate 49 is rotated around the rotating shaft 50 by an urging force generated by the urging members 47 and the contact surface of the stopper 46 is pressed against the spherical surface 45a of the fixed section 45. The urging members 47 according to the present invention are not limited to the helical springs and may be other urging members such as leaf springs.

The moving mechanism 48 is a mechanism for moving the stopper 46 closely attached to the fixed section 45 in a direction away from the fixed section 45 against the urging force of the urging members 47. As the moving mechanism 48, publicly-known various mechanisms can be used. For example, it is possible to use a link mechanism which pushes down a portion on a rear side in the present embodiment of the stopper supporting plate 49 by rotating or moving up and down a lock operation lever 58 provided behind an upper part of the operation section 24.

In the operation section 24, a substrate 51 mounted with electronic components is incorporated. A power switch, a recording switch, a zoom switch, a lighting switch, or the like provided on the side of the operation section 24 is connected to the substrate 51. The substrate 51 is disposed in a position avoiding a position right below the operation lever 42. Specifically, the substrate 51 is attached to a side on an outer side of the frame 43.

The drawing operation member 41 moves the plural operation wires 55 shown in FIG. 6 by performing tilting operation for changing a tilting direction and a tilting angle of the bending lever 40 and bends the bending section 22 in a desired direction by a desired bending amount. The bending section 22 is configured to be substantially linear when the bending lever 40 is in a self-supporting state.

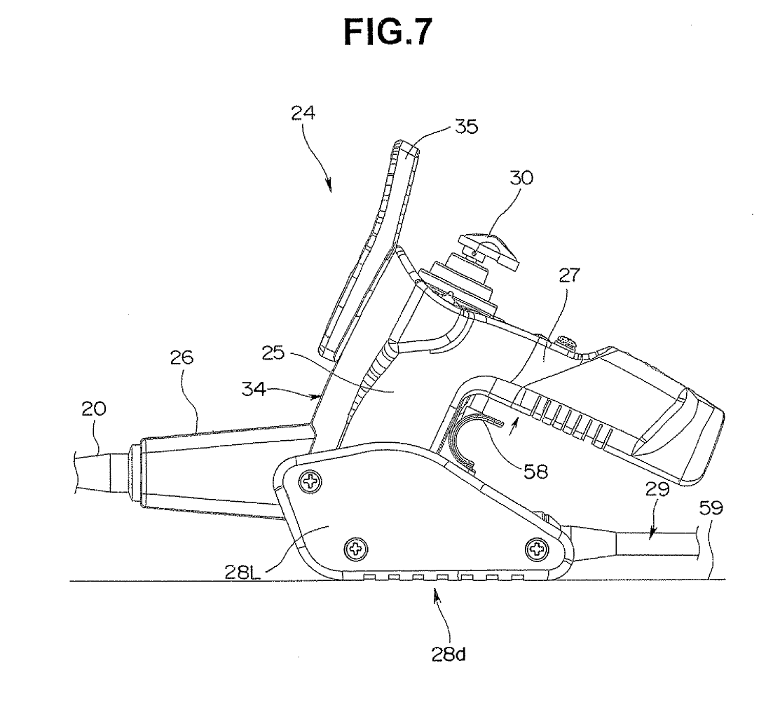

As shown in FIGS. 2 and 7, the operation wire disposing section 26 is protrudingly provided from a front lower surface side of the operation section main body 25. The insertion section 20 extends out from a distal end surface of the operation wire disposing section 26. A longitudinal axis of the operation wire disposing section 26 and an insertion axis of the insertion section 20 are substantially coaxial, in other words, substantially coincide with each other. On the other hand, the grasping section 27 is protrudingly provided from a rear upper surface side to a rear side of the operation section main body 25. A longitudinal axis of the grasping section 27 has an axis different from the insertion axis of the insertion section 20. In other words, the longitudinal axis of the grasping section 27 and the insertion axis of the insertion section 20 do not coincide with each other, i.e., are configured as non-coincident axes. Specifically, as shown in FIG. 7, the longitudinal axis of the grasping section 27 is obliquely set to gradually extend downward from an upper side of the operation section main body 25 rather than horizontally.

In the present embodiment, the drawing operation member 41 is located on a distal end side of the grasping section 27. The universal cable 29 extends out from a rear lower surface side of the operation section main body 25. An extending-out portion of the universal cable 29 is set further on a setting surface side described later than an extending-out portion of the insertion section 20.

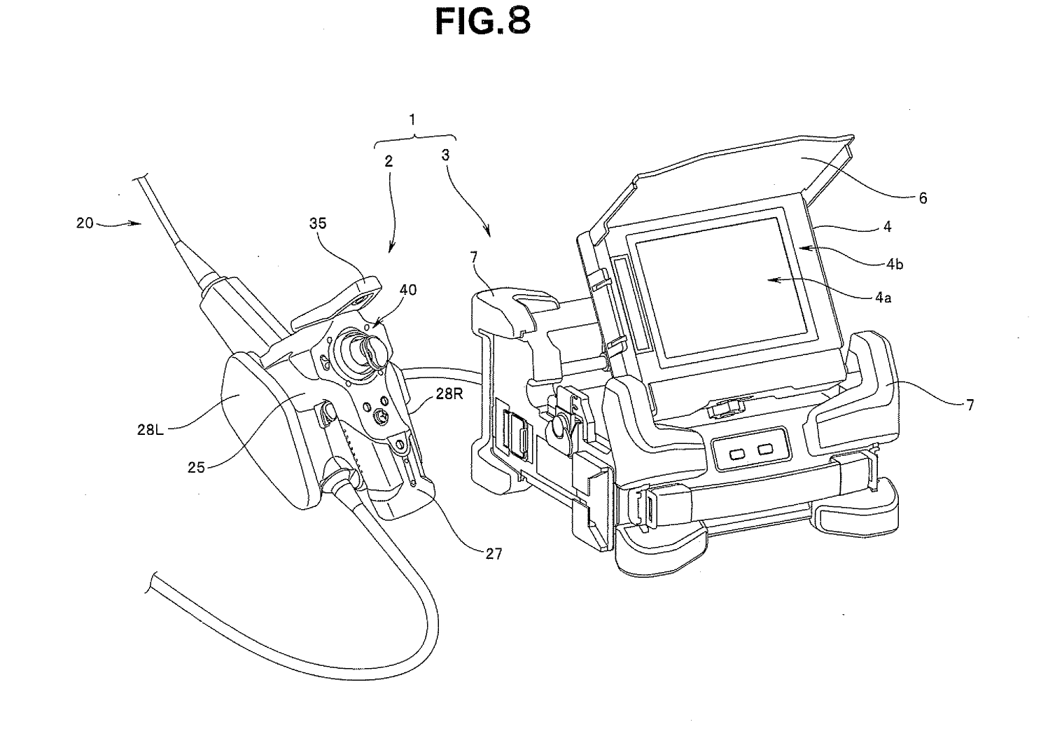

As shown in FIG. 4, the side configuring section 28L is provided on a right side portion of the operation section main body 25 when a front section 34 of the operation section main body 25 configuring the operation section 24 is front-viewed from the insertion section 20 side. On the other hand, the side configuring section 28R is provided on a left side portion of the operation section main body 25. As shown in FIGS. 7 and 8, when the setting surfaces 28d of the side configuring sections 28L and 28R are set on a surface 59 of, for example, a desk or a floor, the operation section 24 is set in vertical placement in which the bending lever 40 faces obliquely upward.

In the vertical placement, since the driving motor 52 and the pulley 53, which are heavy objects, are disposed on the lower side of the operation section main body 25, a center of gravity position of the operation section 24 is set on a lower side of the operation section main body 25 and the operation section 24 can be set on the floor or the like in a stable state.

Since the extending-out portion of the universal cable 29 from the operation section main body 25 is located further on the setting surfaces 28d side than the extending-out portion of the operation section main body 25 of the insertion section 20, the center of gravity position of the operation section 24 is set further on the lower side of the operation section main body 25 and the operation section 24 can be set on the floor or the like in a more stable state. In addition, since the longitudinal axis of the grasping section 27 is obliquely set to gradually extend downward rather than in the horizontal direction, it is possible to set the center of gravity position of the operation section 24 further on the lower side of the operation section main body 25 and make stable setting of the operation section 24 possible.

In the present embodiment, a holding member 35 including a holding section for holding the insertion section 20 is provided in the front section 34 of the operation section main body 25. The holding member 35 is separated from the operation section main body 25 and is integrally fixed to the operation section main body 25 by, for example, an adhesive, screws, or the like. An upper end side of the holding member 35 on an upper side in FIG. 4 is provided to project from the top surface of the operation section main body 25 by a predetermined amount and also serves as a guard member which protects the bending lever 40.

A holding hole 36 is formed in the center in an upper end side portion of the holding member 35 which projects from the top surface of the operation section main body 25. An inner diameter of the holding hole 36 is formed larger than an outer diameter of the insertion section 20 by a predetermined dimension. By inserting the distal end portion 21, the bending section 22, or a distal end side of the flexible tube section 23, which is a distal end side portion of the insertion section 20, through the holding hole 36, the insertion section 20 is held by the holding member 35 and the distal end portion 21 of the insertion section 20 is arranged in the space.

Actions of the endoscope 2 configured as described above are explained.

In performing an endoscopic test using the endoscope apparatus 1 according to the present embodiment, the operator puts a not-shown belt attached to the apparatus main body 3 on, for example, the shoulder, grasps the grasping section 27 of the endoscope 2, and carries the endoscope apparatus 1 to a work place. When the operator arrives at the work place, the operator sets the apparatus main body 3 in a predetermined position and starts the test.

When the operator hands off from the grasping section 27 by mistake while the operator is carrying the endoscope apparatus 1 or when the operator is doing work, the operation section 24 falls from the setting surfaces 28d side of the side configuring sections 28L and 28R, which configure the operation section 24, where the driving motor 52, the pulley 53, and the like, which are heavy objects.

Since the side configuring sections 28L and 28R which collide with the floor or the like are configured to include the bumper members 30L and 30R, a shock at the time of the fall is absorbed by the bumper members 30L and 30R. Therefore, it is possible to protect the substrate 51, the driving motor 52, and the pulley 53 incorporated in the operation section main body 25 from the shock at the time of the fall. Consequently, a failure and the like due to the shock are prevented beforehand.

Since the bumper members 30L and 30R are provided to project further than the outer surface of the outer peripheral portion 31a and the outer surface of the side portion 31b of the operation section housing sides 31L and 31R, the setting surfaces 28d of the side configuring sections 28L and 28R protrude further than the lower surface 25d of the operation section main body 25. Therefore, it is possible to prevent the frame body configuring the operation section main body 25 and the floor or the like from directly colliding with each other and protect the operation section main body 25 from being damaged by the collision. In addition, since the setting surfaces 28d of the side configuring sections 28L and 28R protrude further than the lower surface 25d of the operation section main body 25, it is possible to set the operation section 24 in a stable state avoiding unevenness of a setting place.

Moreover, since the bumper members 30L and 30R are formed by the elastic members such as NBR, it is possible to prevent a slip and set the operation section 24 in a stable state.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An endoscope apparatus comprising:
an operation section main body configured to include a frame body and bumper members provided to cover plural surfaces of the frame body;
an operation section configured to include the operation section main body and a grasping section;
an endoscope including an insertion section extended out from one surface side of the operation section main body and including a bending section in a position more proximal than a distal end and a universal cable extended out from the other surface side of the operation section main body opposed to the one surface; and
an apparatus main body to which the universal cable is connected.

2. The endoscope apparatus according to claim 1, wherein the bumper members configuring the operation section is protrudingly provided on an outer peripheral portion and side portions of the frame body.

3. The endoscope apparatus according to claim 1, wherein
the operation section includes a drawing operation member which bends the bending section,
the drawing operation member is configured to have an indication member which tilts and indicates a bending direction of the bending section, operation wires drawn and relaxed by tilting operation of the indication member, a pulley externally attached with tubular members around which the operation wires are wound, and a driving motor which drives to rotate the pulley, and
the indication member is provided on one side of the operation section main body near a root side of the grasping section, the pulley and the motor are provided on the other side of the operation section main body, and the bumper members are provided on the other side of the operation section main body.

4. The endoscope apparatus according to claim 2, wherein the bumper members are provided on at least two surface of the frame body configuring the operation section, and one surface of the two surfaces configures a setting surface of the operation section.

5. The endoscope apparatus according to claim 1, wherein the universal cable is extended out from the other surface side of the operation section main body and has a connector box at a distal end.

6. The endoscope apparatus according to claim 5, wherein the apparatus main body includes a connector box housing chamber to which the connector box is detachably attachable.

7. The endoscope apparatus according to claim 5, wherein an extending-out portion of the universal cable extended out from the other surface of the operation section main body is located further on a setting surface side than an extending-out portion of the insertion section extended out from one surface of the operation section main body.

8. The endoscope apparatus according to claim 1, wherein the bumper members are shock absorbing members.

9. The endoscope apparatus according to claim 4, wherein
the bumper members, one surface of which configuring a setting surface, are provided on both sides parallel to an insertion section axial direction of the frame body configuring the operation section, and
a space is formed between the setting surface of the bumper members and the setting side surface of the frame body.
